(19) United States
(12) Patent Application Publication
(10) Pub. No.: US 2017/0348271 A1
(43) Pub. Date: Dec. 7, 2017

(54) GLYCERYL LAURATES AND METHODS OF USING THE SAME

(71) Applicant: ACCELERATED GENETIX, LLC, AUSTIN, TX (US)

(72) Inventor: John Dunlop, San Antonio, TX (US)

(73) Assignee: ACCELERATED GENETIX, LLC, AUSTIN, TX (US)

(21) Appl. No.: 15/616,819

(22) Filed: Jun. 7, 2017

Related U.S. Application Data
(60) Provisional application No. 62/347,001, filed on Jun. 7, 2016.

(51) Int. Cl. A61K 31/23; A61K 9/48; A61K 9/00

(52) U.S. Cl. CPC A61K 31/23; A61K 9/0053; A61K 9/48

(57) ABSTRACT

A method of promoting muscle growth consisting of providing a dietary supplement containing an effective amount of glyceryl laurates to a user. A composition for promoting muscle growth containing an effective amount of glyceryl laurates.

### CROSS-REFERENCE TO RELATED APPLICATIONS

This application claims the benefit of U.S. Provisional Application No. 62/347,001, filed on Jun. 7, 2016, the teachings of which are expressly incorporated by reference.

### STATEMENT RE: FEDERALLY SPONSORED RESEARCH/DEVELOPMENT

Not Applicable

### BACKGROUND

The present disclosure relates generally to a dietary supplement, which promotes muscle enlargement, and more particularly to a dietary supplement containing glyceryl laurates.

It is well known that a person's diet affects their good health and quality of life. It is important to intake the necessary nutrients for a healthy lifestyle. While a person's diet generally determines which nutrients they acquire, dietary supplements may be consumed to supplement nutrients lacking in a person's diet. Dietary supplements are food products that supply nutrients that may be missing or not consumed in sufficient quantity. The food product may include one or more of a vitamin, mineral, herb or other botanical, or an amino acid. The food product can also include a dietary substance to supplement the diet by increasing a person's total dietary intake. More information regarding dietary supplements can be found in the Dietary Supplement Health and Education Act of 1994.

In general, the nutrients a person requires depends on many different factors, such as their activity level. Some people need and benefit from nutrients that promote muscle enlargement. One way of accomplishing this is by the administration of glycerol. Glycerol is sometimes also referred to as glycerine or glycerin, and all three names are scientifically accepted and interchangeable.

Glycerol (see FIG. 1) is a colorless, odorless, simple polyol (sugar alcohol) compound. It is a viscous, non-toxic liquid with a sweet taste and sticky tactile character. It is widely used in the food industry as a sweetener and humectant, and in numerous pharmaceutical formulations. Glycerol has three hydroxyl functions on its molecular backbone that make it not only hygroscopic (water-soluble) in nature, but also provide three locations that can be chemically esterified. The glycerol backbone itself is central to all lipids known as fatty acid glycerides.

Being a hygroscopic liquid, glycerol is easy to add to commercial preparations designed as rehydrating liquids for athletes or those suffering from conditions of dehydration. However, the liquid is not convenient for dry formulations such as pills and powders. Therefore, a chemical compound incorporating glycerin into its chemical structure could be used as a pro-form of glycerin, if it's structure was a stable solid from which glycerol could be liberated after ingestion (in-vivo.) Such liberation is known as hydrolysis, and occurs in the body after ingestion of fatty acid glycerides. Upon hydrolysis, glycerol laurates becomes glycerin and lauric acid(s). Monolaurin, also known as glycerol monolaurate, glyceryl laurate, 1-lauroyl-glycerol, and glycerol lauric acid ester, (see FIG. 2) is a fatty acid glyceride compound with the properties needed to match this useful specification. As mentioned prior, the glycerol molecule can accommodate three additions and the di- and tri-laurate esters of glycerol also meet the useful criteria, but in descending order of efficiency from the ideal mono-laurate ester of glycerol.

Monolaurin has been used for many things related to its hydrolysis product lauric acid, which possesses properties making it a useful biological substrate. However, glyceryl laurates like monolaurin have not previously been utilized for benefit of their glycerin component. Monolaurin has been used for its reputed plethora of antimicrobial and emulsifying applications in the area of human food products, but never as a non-toxic, stable, dry glycerol donor.

Glycerol monostearate is another fatty acid ester of glycerol composed of glycerol and stearic acid upon hydrolysis. It has been, and still is, utilized as an in-vivo glycerol donor. However, due to widespread popular belief and compelling biological evidence that stearic acid has the potential for detrimental immune system effects, its use has declined. This has created market demand for a more safe and efficacious replacement.

As such, there is a need for a safe and effective glycerol donor agent that does not detrimentally affect the user's immune system. In that regard, lauric acid demonstrates none of the detrimental potentials of stearic acid, and in fact is used extensively to promote a healthy immune condition in the body. Therefore, monolaurin and its other related glyceryl laurates offer a superior form of fatty acid glycerol donor. Additionally, monolaurin has a lower molecular mass than stearates, and thus provides a greater relative potency and glycerol contribution in comparison to stearates. Monolaurin provides the intended benefit without the toxicity concerns of stearates and silicates previously known and used in the art. And, further, monolaurin is capable of being used in a form applicable to non-liquid preparations when glycerol itself is not practical.

### BRIEF SUMMARY

In accordance with one embodiment of the present disclosure, there is contemplated a method of increasing muscle enlargement in a user in need of said muscle enlargement. The method includes providing to the user a composition having an effective amount of at least one glyceryl laurate. The composition may contain at least one ester of glyceryl laurate.

The composition may further include a carrier which carries the glyceryl laurate. The carrier may be a solid, a liquid, or a combination of the two. The carrier may be shaped and dimensioned to facilitate its oral ingestion.

The composition may be provided to the user once per day, or multiple times throughout the day. The composition may be provided to the user such that the user is provided between approximately 0.5 g and approximately 10 g daily of glyceryl laurate. The user may be provided approximately 2.6 g daily of the glyceryl laurate.

In accordance with another embodiment of the present disclosure, there is contemplated a method of increasing muscle enlargement in a user in need of said muscle enlargement. The method includes providing to the user an effective amount of a glyceryl laurate. The glyceryl laurate may be combined with a solid or liquid carrier. The glyceryl laurate may be provided in more than one compositional form. In particular, the glyceryl laurate may have the lauric acid attached to a functional group of the glycerol at one, any, or all reactive points of the compound. In accordance with yet another embodiment of the present disclosure, there is contemplated a composition for increasing muscle enlargement in a user. The composition includes an effective amount of at least one glyceryl laurate. In particular, the composition may include an ester of glyceryl laurate.

The composition may further include a carrier. The carrier may be a solid, a liquid, or combinations of the two. The carrier may be shaped and dimensioned to facilitate its oral ingestion.

The composition may include more than one compositional form of glyceryl laurate. The composition may include at least 90% glycerol monolaurate and less than 10% combined glycerol dilaurate and glycerol trilaurate.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a skeletal formula of glycerol;

FIG. 2 is a skeletal figure of glycerol monolaurate; and

FIG. 3 is a chemical formula of glycerol, showing the three positions naturally occupied by a hydrogen atom which can be substituted for an organic acid by esterification.

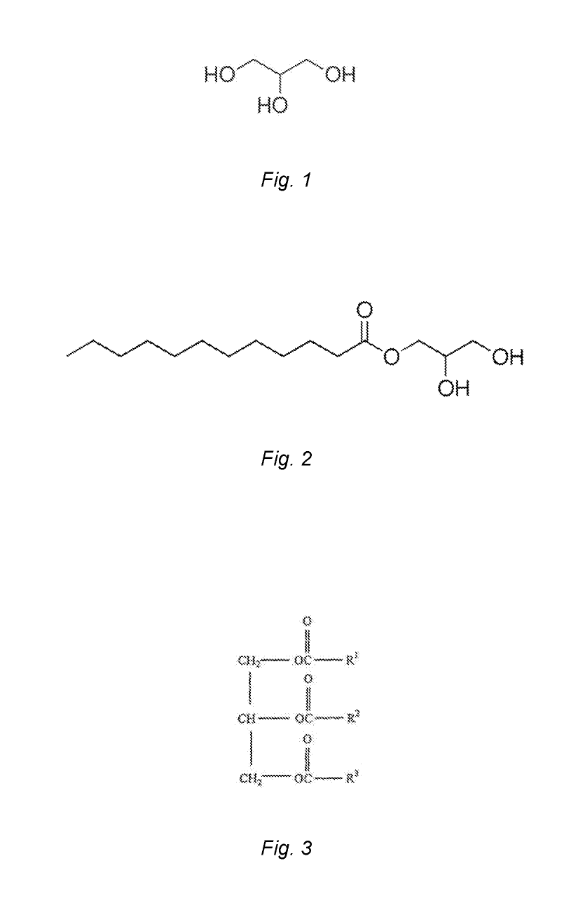

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

One embodiment of the present disclosure comprises a composition including at least one glycerol ester (glyceride) of lauric acid(s) (systematically, dodecanoic acid), simply known as a glyceryl laurate. If referring specifically to the monoglyceride of lauric acid, monolaurin is the preferred form. Monolaurin can also be accurately referred to as a glyceryl laurate or 1-lauroyl-glycerol.

The composition contains an effective amount of glyceryl laurates to effect muscle growth in a user. The composition may include several compositional forms of glyceryl laurates. For example, the use of lauric acid glycerides other than the monoglyceride (such as the di- and tri-glycerides of lauric acid) are not precluded as formulary options, and may also be utilized depending on manufacturing variables and product specifications. The composition may also include isomers, stereoisomers, or isosteres of glyceryl laurates. In particular, FIG. 3 shows the possible inclusions and positions of substitution on the natural glycerol molecule.

The composition may be contained within a carrier, wherein the carrier may be a solid or liquid. The invention is preferably ingested orally, so its size and shape may be chosen to facilitate oral ingestion. The composition may be contained in the form of a pill, tablet, soft-gel, pearl, caplet, capsule, and the like. In a preferred embodiment, the composition may be contained within a gelatin and/or vegetable capsule, which will all be henceforth consolidated into the term “gel cap.” The composition may also be in the form of a powder, so it can be later combined with a liquid carrier prior to use.

The composition may be taken by the user in a single bolus dose, or multiple doses divided appropriately throughout a twenty-four hour period (daily.) The active content of the composition (triglycerides, diglycerides, and/or monoglycerides of lauric acid) may also further include excipients and/or other ingredients within the carrier. The total daily dose may be variable, depending on calculations based mainly on the weight of the user, but would generally be between 0.5 g and 10 g daily. However, other doses are not excluded and may be appropriate for situations such as the initial loading phase of use, or other special applicatory considerations. Just like other mono-, di-, and tri-glycerides of all edible fatty acids, the composition may be added to foods for supplemental value. For example, coconut oil is a common food additive consisting of up to 50% glycerol monolaurate, with similar physical characteristics and food augmenting potential as the composition described herein.

### Human Clinical Evaluations

Six test subjects volunteered to use gel caps filled with the composition. The test material consisted of not less than 90% glycerol monolaurate and not more than 10% combined glycerol dilaurate and glycerol trilaurate with traces of hydration and free glycerol. The free acid value of the material was nil. 78 g of test material (equating to 120 gel caps of 650 mg contents each) were given to each tester, and testers were advised to use 2-6 doses daily as they saw fit until they ran out of supplied material. Test subjects were also required not to use other supplementations, which could skew interpretation of the results. All subjects complied with the criteria, and used an average 2.6 g of test material daily.

At the end of testing all subjects presented similar results. Namely, their muscles became hypertrophic in response to physical movement combined with ingestion of the test material. The most profound benefits were experienced with weight/resistance training or when performing aerobic exercises.

All tests subjects agreed that the sensation of muscle perfusion (also known as “pump”) began approximately 30-60 minutes after oral ingestion, and persisted for up to 24 hours post dosing. All subjects reported experimenting with 1 to 4 divided doses daily, with the intensity of effect being demonstrating positive dose-dependent linearity. In other words, higher doses offered exaggerated effects, which were sometimes appropriate depending on the muscle group being trained.

The test subjects were warned to discontinue testing if any side effects appeared that they suspected may be related to the testing regimen. Nevertheless, no subjects abandoned testing and none reported side effects such as headache, altered blood pressure, gastrointestinal complications, or undesirable collateral bloat in tissues adjacent to muscle.

It is not clearly understood why glyceryl laurates demonstrate this muscle enlarging ability, and the invention is not intended to be limited by any theories, but a hypothesis is presented as follows. It is known that glycerol is quite hygroscopic, and thus in the body encourages water retention to create a state of hyper-hydration. It is also known that hyper-hydration necessarily increases blood volume, which accounts for enhanced perfusion. Perfusion is the process of blood delivery to the capillary beds in humans, and this mechanism is also the prime regulator of capillaries in skeletal muscle. Adaptation of blood perfusion in muscle occurs according to body demand, which likely explains the high specificity of effect the test athletes reported in muscle growth, without change in healthy cardiovascular parameters. The glycerol released from the lauric ester of the invention may end up being used in metabolism, which results in local deposition of the hydration to which it was chemically bonded. This is the location of adaptation, and thus the muscle would be enlarged. It is also inevitable that increased tissue oxygenation results with increased perfusion, thus also explaining the accelerated metabolism and growth in affected tissues. All test subjects reported semi-permanent gains in muscle size, appearance and strength, even weeks after the clinical trials were concluded.

The above description is given by way of example, and not limitation. Given the above disclosure, one skilled in the art could devise variations that are within the scope and spirit of the invention disclosed herein, including various ways of dosing the composition and carriers for providing the composition to users. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of increasing muscle enlargement in a user in need of said muscle enlargement, said method comprising:
providing to the user a composition comprising an effective amount of at least one glyceryl laurate.

2. The method of claim 1, wherein the composition comprises at least one ester of glyceryl laurate.

3. The method of claim 1, wherein the composition further comprises a carrier which carries the at least one glyceryl laurate.

4. The method of claim 3, wherein the carrier is selected from the group consisting of a solid, a liquid, and combinations thereof.

5. The method of claim 3, wherein the carrier is shaped and dimensioned to facilitate its oral ingestion.

6. The method of claim 1, wherein the composition is provided to the user once per day.

7. The method of claim 1, wherein the composition is provided to the user such that the user is provided between approximately 0.5 g and approximately 10 g daily of the at least one glyceryl laurate.

8. The method of claim 7, wherein the user is provided approximately 2.6 g daily of the at least one glyceryl laurate.

9. A method of increasing muscle enlargement in a user in need of said muscle enlargement, said method comprising:
providing to the user an effective amount of a glyceryl laurate.

10. The method of claim 6, wherein the glyceryl laurate is combined with a solid or liquid carrier.

11. The method of claim 6, wherein more than one compositional form of glyceryl laurate is provided to the user.

12. The method of claim 6, wherein the glyceryl laurate has the lauric acid attached to a functional group of the glycerol at one, any, or all reactive points of the compound.

13. A composition for increasing muscle enlargement in a user, said composition comprising an effective amount of at least one glyceryl laurate.

14. The composition of claim 10, wherein the composition comprises at least one ester of glyceryl laurate.

15. The composition of claim 10, wherein the composition further comprises a carrier.

16. The composition of claim 12, wherein the carrier is selected from the group consisting of a solid, a liquid, and combinations thereof.

17. The composition of claim 12, wherein the carrier is shaped and dimensioned to facilitate its oral ingestion.

18. The composition of claim 10, wherein the composition comprises more than one compositional form of glyceryl laurate.

19. The composition of claim 10, comprising at least 90% glycerol monolaurate and less than 10% combined glycerol dilaurate and glycerol trilaurate.
